(19) United States
(12) Patent Application Publication
(10) Pub. No.: US 2009/0028798 A1
(43) Pub. Date: Jan. 29, 2009

(54) METHOD FOR DETERMINING REDOX STATUS OF A TISSUE

(75) Inventors: James B. Mitchell, Damascus, MD (US); Murali K. Cherukuri, North Potomac, MD (US); John A. Cook, Bethesda, MD (US); Fuminori Hyodo, Anjo Aichi (JP); Alan P. Koretsky, Bethesda, MD (US); Ken-Ichiro Matsumoto, Chiba (JP); Sankaran Subramanian, Rockville, MD (US); David A. Wink, Hagerstown, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: Government of the USA, represented by the Secretary, Dept.of Health and Human Services, Rockville, MD (US)

(21) Appl. No.: 11/997,236

(22) PCT Filed: Aug. 10, 2006
(86) PCT No.: PCT/US06/31208

§ 371 Date: Feb. 19, 2008

Related U.S. Application Data
(60) Provisional application No. 60/707,518, filed on Aug. 11, 2005.

(51) Int. Cl. A61K 49/08

(52) U.S. Cl. 424/9.33

(57) ABSTRACT

Disclosed is a method for determining the redox status of a region of interest in an animal tissue. The method includes administering a nitroxyl contrast agent to the region of interest, obtaining a magnetic resonance image of the region of interest, determining the amount of reduced nitroxyl contrast agent in the region of interest, and thereby determining the redox status of the region of interest.

### BACKGROUND OF THE INVENTION

This invention relates to magnetic resonance imaging (MRI) of an animal tissue employing nitroxyl contrast agents.

The use of MRI to monitor tissues, particularly, tumors, is known. MRI measures the size of a solid tumor, and a change in size of the tumor indicates whether the tumor has been affected by a cancer treatment (i.e., chemotherapy, radiation therapy). MRI has a number of advantages, for example, it is non-invasive and provides useful anatomical information on tissues. However, presently available MRI techniques do not adequately provide more fundamental information of the tissue, particularly on the chemical nature of the tissue (such as oxidation-reduction or “redox” status) which is indicative of the susceptibility of the tissue to radiation damage or treatment.

The foregoing shows that there is a need for a method of determining the redox status of a tissue of interest, particularly a tumor tissue. The invention provides such a method. These and other advantages of the invention, as well as additional inventive features, will be apparent from the description of the invention provided herein.

### BRIEF SUMMARY OF THE INVENTION

The invention provides a method for determining the redox status of a region of interest in an animal tissue comprising: a) administering a nitroxyl contrast agent to the region of interest, b) obtaining a magnetic resonance image of the region of interest, and c) determining the amount of reduced nitroxyl contrast agent in the region of interest and determining the redox status of the region of interest.

The invention also provides a method for diagnosing a tumor in a region of interest in an animal tissue comprising: a) administering a nitroxyl contrast agent to an animal tissue whose region of interest is to be monitored, b) obtaining a magnetic resonance image of the region of interest, c) obtaining a magnetic resonance image of a tissue adjacent to a region of interest, d) determining the amount of reduced nitroxyl contrast agent in the tissue adjacent to the region of interest, e) determining the amount of reduced nitroxyl contrast agent in the region of interest and determining the redox status of the tissue adjacent to the region of interest relative to the redox status of the region of interest, and f) diagnosing whether there is a tumor present based on the redox status of the region of interest.

Also provided by the invention is a method for determining a cancer treatment protocol comprising: a) administering a nitroxyl contrast agent to a subject with a tumor, b) obtaining a magnetic resonance image of the tumor, c) obtaining a magnetic resonance image of a tissue adjacent to the tumor, d) determining the amount of nitroxyl contrast agent in the tumor, e) determining the amount of nitroxyl contrast agent in the tissue adjacent to the tumor, and f) determining the difference in the amount of nitroxyl contrast agent in the tumor compared with the amount of nitroxyl contrast agent in the tissue adjacent to the tumor to determine a time suitable to administer a dose of radiation. The invention also provides a method of cancer treatment by radiotherapy based on this.

### BRIEF DESCRIPTION OF THE SEVERAL VIEWS OF THE DRAWINGS

FIG. 1 is a series of graphs comparing the nitroxyl radical decay rate, determined by the change in contrast in MRI over time, of Tempol, Carbamoyl-PROXYL, and Carboxyl-PROXYL in various tissues over time.

FIG. 2 is a graph of T1 contrast change (Y-axis on the left) and total nitroxide volume (Y-axis on the right) which compares the redox status, determined by the change in contrast in MRI over time, between a tumor and normal tissue using in vivo MRI and nitroxyl contrast agent Tempol, in accordance with the present invention.

FIG. 3 is a graph showing the change in the electron paramagnetic resonance (EPR) signal intensity in a normal leg and a tumor leg, over time.

FIG. 4 is a graph of the decay profiles of a nitroxyl contrast agent Carbamoyl-PROXYL (3CP), in a normal leg and a tumor leg observed by EPR spectroscopy.

FIG. 5 is a graph of the time course of the change in contrast signal intensity of 3CP over time in a normal leg and a tumor leg, by MRI, in accordance with the present invention.

### DETAILED DESCRIPTION OF THE INVENTION

Tumor tissues exhibit viable but hypoxic regions that allow them to reduce nitroxides more efficiently than normal tissue. The present invention is predicated on the difference in reducing capability and provides a method of determining the redox status of a region of interest in an animal tissue, such as a tumor. By determining the redox status of a tumor it is possible to not only diagnose a tumor due to its enhanced reduction of intracellular nitroxide contrast agent, but also to determine appropriate radiation treatment fields spatially to deliver therapeutic doses of radiation, and to determine appropriate timing sequences after the administration of a nitroxide contrast agent such that the maximum difference between normal and tumor tissue with respect to the radioprotective form of the nitroxide is present in the normal tissue, thereby limiting collateral damage to the normal tissue. The T1-contrast afforded by the nitroxide class of compounds, by virtue of their paramagnetic relaxivity which is in the range of 0.2 (mM s)−1 makes it possible to use standard MRI scanners to obtain the redox information in the inventive method. Typical relaxivity and relaxation times are shown for various nitroxyl contrast agents compared with a standard contrast agent, Gd-DTPA, in Table 1. MRI contrast shows excellent anatomical mapping based mainly, on spin density, T1 and T2 of water proton. Without being bound to any particular theory, it is believed that the T1 relaxation of protons could be affected by paramagnetic electron spin. Therefore, a change of MRI contrast before and after administration of a nitroxyl spin probe (i.e., a nitroxide contrast agent) should reflect the amount of nitroxyl in addition to providing anatomical mapping simultaneously. Such anatomical mapping provides the ability to diagnose the existence of a tumor, determine the status of a tumor, determine borders of a radiation treatment field, determine appropriate timing and dosage for radiation treatment, as well as determining the efficacy of radiation and other forms of cancer treatment.

TABLE 1

| | Relaxation | |
|---|---|---|
| Contrast Agent | Time (sec) | Relaxivity s · mM⁻¹ |
| 15N-d16-Tempone | 2.198 | 0.166 |
| | 1.993 | |
| | 1.888 | |
| | 1.733 | |
| | 1.515 | |
| | 1.336 | |
| Tempone | 2.168 | 0.171 |
| | 1.995 | |
| | 1.846 | |
| | 1.706 | |
| | 1.475 | |
| | 1.313 | |
| Carbamoyl-PROXYL | 2.152 | 0.171 |
| | 1.915 | |
| | 1.773 | |
| | 1.624 | |
| | 1.406 | |
| | 1.231 | |
| Carboxy-PROXYL | 2.362 | 0.180 |
| | 2.170 | |
| | 1.966 | |
| | 1.813 | |
| | 1.660 | |
| | 1.285 | |
| Tempol | 2.152 | 0.180 |
| | 1.951 | |
| | 1.831 | |
| | 1.683 | |
| | 1.465 | |
| | 1.299 | |
| Gd-DTPA | 0.485 | 4.862 |
| | 0.920 | |

Accordingly, in one embodiment, the invention provides a method of determining the redox status of a region of interest in an animal tissue. The method includes administering a nitroxyl contrast agent to a region of interest, obtaining a magnetic resonance image of the region of interest, determining the amount of reduced nitroxyl contrast agent in the region of interest and determining the redox status of the region of interest.

The nitroxyl contrast agent (also referred to herein as a nitroxide), can be any nitroxide that permeates the cell membrane and therefore accumulates intracellularly. Suitable nitroxide contrast agents include, but are not limited to, 3-carbamoyl-2,2,5,5-tetramethyl-1-pyrrolidine-N-oxyl (Carbamoyl-PROXYL, ie. “3CP”), 3-carboxy-2,2,5,5-tetramethyl-1-pyrrolinyloxy (Carboxy-PROXYL), 2,2,6,6-tetramethyl-4-piperidinol-N-oxyl (Tempol), N-d16-triacetoneamine-N-oxyl (N-d16-Tempone), and triacetonamine-N-oxyl (Tempone).

The method is useful in determining the redox status of a region of interest (ROI) in an animal tissue. Preferably the animal is a human. The region of interest can be a part of a tissue or the whole tissue. The region of interest can be any shape such as circular, square triangular, trapezoidal, and any area such as 0.1 mm² to 100 mm², 1 mm² to 10 mm², 2 mm² to 6 mm², or more. The region of interest can be defined in a normoxic or hypoxic tissue

A hypoxic tissue may be hypoxic due to a variety of conditions. One example of a hypoxic tissue is a tumor tissue. A tumor tissue includes any form of solid tumor that includes a hypoxic but viable region. The tumor can be located anywhere in the body and can be of any grade (ie. I-IV, low, mid, high, etc.), origin, or size. For example the tumor can be located in any organ or gland of the body including but not limited to the brain, lung, stomach, liver, pancreas, gall bladder, small intestine, large intestine, kidney, integumentary, bone, ovary, uterus, cervix, prostate, testicle, bladder, mouth, throat, thyroid, adrenal gland, pituitary, head, neck, brain stem, spinal cord, etc. The tumor can be a primary tumor or a metastastic tumor.

The normoxic tissue can be any tissue exhibiting a normal redox status. The normoxic tissue can be a tissue that is adjacent to a tumor. Further, the normoxic tissue can be adjacent to a tumor and can be at risk of infiltration by the tumor. The normoxic tissue can further be a tissue that is within the field of radiation treatment. Alternatively, the normoxic tissue can be situated in a location that is not adjacent to a tumor and can serve as a control against which the redox status of the tumor tissue is compared.

In the inventive method, magnetic resonance image (MRI) is obtained of an region of interest (ROI) in an animal tissue, i.e., a normoxic tissue, a hypoxic tissue or both. An image of the tissue can be obtained prior to, at the time of, and/or after administration of the nitroxyl contrast agent. Further, more than one image can be obtained of each tissue after the administration of the nitroxyl contrast agent. That is, images can be obtained over a period of time to determine the accumulation and clearance profile of the nitroxyl agent in the tissue of interest, Multiple images obtained as a function of time can provide useful information regarding the radioprotective status of the normoxic and/or hypoxic tissue. For example, images can be obtained over a period of time such as 1, 5, 10, 20, 30, 40, or 60 minutes, or any integer in between, after administration of a nitroxyl contrast agent. Images can be taken at intervals of every few seconds or minutes.

In the inventive method, the amount of reduced nitroxyl contrast agent (i.e., hydroxylamine) in the animal tissue is determined. This determination can be made using any suitable method. For example, MRI contrast changes and the difference in concentration of a nitroxyl contrast agent, such as 3CP, show linearity in low concentration level (i.e., below 1.5 mM). To perform T1 and T2 mapping, spin echo images can be obtained using a multi-slice multi-echo (MSME) sequence. For instance, a time sequence of the total number of SPGR images may be determined. The images can then be averaged and each image may be divided by the averaged initial image. Semi-logarithmic values of the averaged image intensity in a given region of interest can be plotted versus time after injection. The change in intensity can be determined at one or more time points after injection of a nitroxyl contrast agent. For example, changes in intensity may be made every 10 s, 20 s, 30, 40 s, 50 s, or every 1, 5, 7, 10, 15, 20 minutes, or any integer in between, after injection of a nitroxyl contrast agent. A decay rate can be obtained from the linear part of the slope after peak by the least squares method. A sample calculation of a decay rate using nitroxyl contrast agent 3CP is as follows: The spoiled gradient echo (SPGR) image intensity Mt at time t after nitroxyl contrast agent injection can be calculated using the equation:

Mt=M0×[1−EXP(−TR/T1t)]×EXP(−TE/T2)×{sin α/[1−cos α×EXP(−TR/T1t)]}.

In the above equation, MO is proton density, and a is flip angle. Tissue (or sample) T1 can change with particular time t (min.) (T1t) depending on the concentration of the 3CP (Ct). T1t<1/R1t and R1t=1/T1i+r1×Ct, where R1t is T1 relaxation time t, the relaxivity r1 of 3CP is 0.17 mM^{-1s-1}, T1i is the initial T1 base line (intrinsic tissue T1). Ct (mM), which is the first concentration of 3CP at particular time t (min.), is calculated by assuming first order decay as indicated by the equation Ct=Cmax×EXP(=ktrue×t), where ktrue is the given decay rate. Logarithmic values of the intensity change from the baseline (ie. ΔM % t=(Mt/Mi−1)×100 or ΔMt=Mt−Mi), can be plotted with time t. Mi is the intrinsic signal intensity of the tissue (or sample) calculated as Ct=0. The decay constant kMRI can be obtained from the slopes of the plots ΔM % t and ΔMt by least square fit.

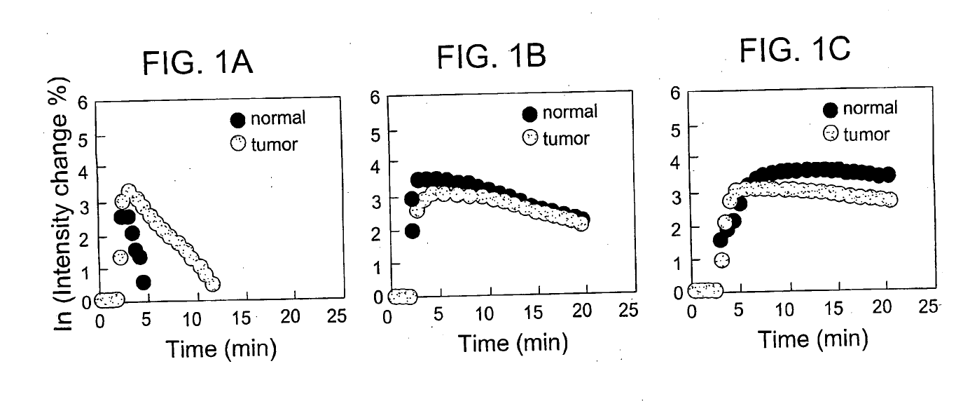

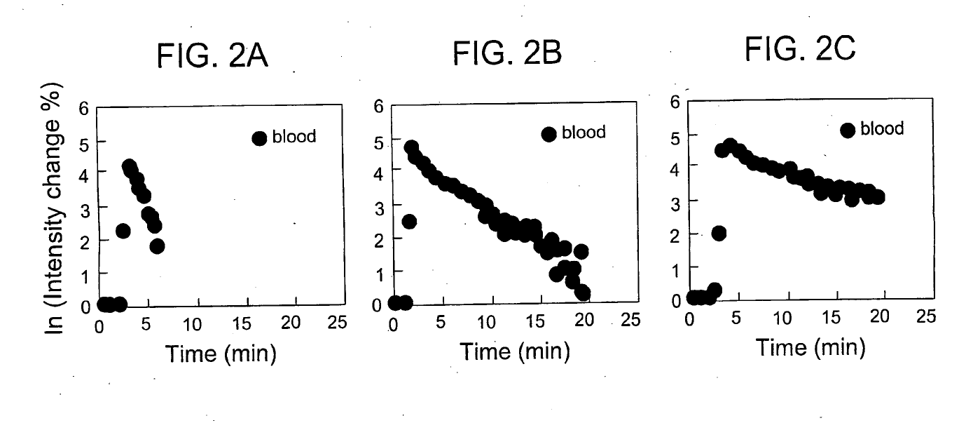

Typical decay rates, determined by a change in intensity over time, are shown in Table 1 and FIG. 1 for nitroxyl contrast agents Tempol, Carbamoyl-PROXYL, Carboxyl-PROXYL in normal tissue, tumor, blood, and left and right kidney tissues. These decay rates may then be used to determine the redox status of the tissue. When a decay rate is calculated pixel-wise, redox mapping can be obtained. That is, regions showing a faster rate of decay correspond to hypoxic regions. FIG. 2 shows a comparison of redox status, as determined by the change in intensity as a function of time, between a normal tissue and a tumor tissue using in vivo MRI and the nitroxyl contrast agent Tempol. As shown, the rate of decay is greater in tumor versus normal tissue.

As discussed, the amount of reduced nitroxyl contrast agent in the tissue is used to determine the redox status of the tissue. The redox status can be determined for normoxic tissue, hypoxic tissue, or both. Preferably, the redox status of a hypoxic tumor tissue and a normoxic tissue adjacent to the tumor are determined. Preferably, the redox status of the tissue is determined by comparing the images obtained at one or more time points after administration of the nitroxyl contrast agent. The image or images obtained correspond to the amount of reduced and non-reduced nitroxyl contrast agent in the tissue and are therefore correlated to the redox status of the animal tissue.

TABLE 2

| | | Carbamoyl- | |
|---|---|---|---|
| | Tempol | PROXYL | |
| | Decay | Decay | Carboxy-PROXYL |
| Tissue | Rate (min⁻¹) | Rate (min⁻¹) | Decay Rate (min⁻¹) |
| Normal Leg | 0.319 ± 0.025 | 0.056 ± 0.013 | 0.029 ± 0.014 |
| Tumor Leg | 1.095 ± 0.203** | 0.107 ± 0.020* | 0.020 ± 0.014 |
| Blood | 1.025 ± 0.213 | 0.364 ± 0.008 | 0.352 ± 0.162 |
| Left Kidney | 1.470 ± 0.199 | 0.304 ± 0.046 | 0.046 ± 0.006 |
| Right Kidney | 1.160 ± 0.333 | 0.294 ± 0.044 | 0.050 ± 0.005 |

Values are indicated as mean ± SD. Significances between the normal leg and the tumor leg are indicated by *= p < .05 and **= p < 0.01.

Any suitable MRI techniques can be utilized in the present invention. In a preferred embodiment, the spoiled gradient echo (SPGR) MRI techniques are employed.

In another embodiment, a method for diagnosing a tumor in a region of interest in an animal tissue is provided. The method includes administering a nitroxyl contrast agent to an animal whose region of interest is to be monitored, obtaining a magnetic resonance image of the region of interest, obtaining a magnetic resonance image of a tissue adjacent to the region of interest; determining the amount of reduced nitroxyl contrast agent in the tissue adjacent to the region of interest, and determining the amount of reduced nitroxyl contrast agent in the region of interest. Further, the amounts of reduced nitroxyl contrast agent are used to determine the redox status of the region of interest relative to the redox status of the region of interest. The redox status information is then used in diagnosing whether there is a tumor present in the region of interest.

In yet another embodiment, a method of cancer treatment by radiation therapy is provided. The method includes administering a nitroxyl contrast agent to an animal tissue, obtaining a magnetic resonance image of a region of interest in the animal tissue, determining the amount of reduced nitroxyl contrast agent in the region of interest and determining the redox status of the region of interest. Then a determination is made as to the time to administer a dose of radiation to the region of interest. The region of interest can be a tumor, normal tissue adjacent to the tumor or both. That is, the tumor and normal tissue adjacent to the tumor that is at risk of being infiltrated by the tumor and is therefore within the field of radiation treatment. The method can further include determining a time when the tissue adjacent to a tumor is most protected from radiation therapy by determining when the tissue adjacent to the tumor contains the greatest concentration of nitroxyl contrast agent. The method can further include determining the boundaries between a tumor and the tissue to the tumor and administering radiation therapy accordingly.

The redox statuses of the normoxic and/or hypoxic tissues can be utilized in developing a cancer treatment protocol. For instance, redox information can be used to determine the appropriate time to administer a dose of radiation to a tumor tissue. For instance, a time that corresponds to greatest amount of nitroxyl contrast agent within the normoxic cell and the greatest amount of reduced nitroxyl contrast agent within the hypoxic cell can be determined. Therefore, a dose of radiation can be administered at such a time to minimize the degree of collateral damage to the normoxic tissue and at the same time, the greatest degree of effectiveness against the hypoxic tumor tissue. Further, the images obtained and redox statuses determined can be used to determine if a tumor has grown or been reduced in size following a form of cancer treatment, such as radiation therapy, chemotherapy, or a combination thereof. Therefore, the inventive method provides a noninvasive means of assessing the status of a tumor and the efficacy of a cancer treatment regimen.

In another embodiment, there is provided a method for determining a cancer treatment protocol that includes administering a nitroxyl contrast agent to a subject with a tumor, obtaining magnetic resonance images of the tumor and tissue adjacent to the tumor, determining the amount of nitroxyl contrast agent in the tumor, determining the amount of nitroxyl contrast agent in the tissue adjacent to the tumor, and determining the difference in the amount of nitroxyl contrast agent in the tumor compared with the amount of nitroxyl contrast agent in the tissue adjacent to the tumor to determine a suitable time to administer a dose of radiation. Preferably, the dose of radiation is administered when there is the greatest difference in the amount of reduced nitroxyl contrast agent in the tumor compared to the tissue adjacent to the tumor. The subject can be an animal and is preferably a human. Stable nitroxide species, which are cyclic organic free radicals, have been shown to provide selective radioprotection to normal tissues (Mitchell, Biochem. Biophys., 289, 62-70 (1991)), while not having any radiation modifying effect on tumors. Experimental observation suggest that the selective protection afforded to normal tissue against the lethal effects of ionizing radiation is due to a more efficient conversion of nitroxide species to its reduced hydroxylamine form in tumors compared to normal tissue (Mitchell, Mil. Med., 167, 49-50 (2002)). Tumors exhibit hypoxic regions and nitroxides are reduced more rapidly under hypoxic conditions. Therefore, knowledge regarding the difference in nitroxyl contrast agent within a normal tissue and a tumor tissue is useful in determining when to administer a dose of radiation. That is, preferably, a dose of radiation is administered when the normal tissue contains the greatest amount of nitroxyl contrast agent in its non-reduced form, and the tumor tissue contains the greatest amount of reduced nitroxyl contrast agent. In this way, the normal tissue will be afforded protection from the damaging effects of radiation and the tumor tissue will be most susceptible to the effects of radiation therapy.

The following example further illustrates the invention but, of course, should not be construed as in any way limiting its scope.

### EXAMPLE

This example demonstrates the effectiveness of using a nitroxyl contrast agent in an MRI based imaging protocol for determining redox status of a tissue, in accordance with an embodiment of the invention.

Materials and Methods. Carbamoyl-PROXYL (3-carbamoyl-2,2,5,5-tetramethylpyrrolidine-N-oxyl: 3CP) was purchased from Sigma-Aldrich Chem. Co. (St. Louis, Mo.). Deionized water (deionization by the Milli-Q system) was used for all experiments. Other materials used were of analytical grade. 3CP was prepared as 300 mM isotonic solution in deionized water.

Female C3H mice were supplied by the Frederick Cancer Research Center, Animal Production (Frederick, Md.). Animals, received at six weeks of age, were housed five per cage in climate controlled circadian rhythm-adjusted rooms and were allowed food and water ad libitum. Experiments were carried out in compliance with the Guide for the Care and Use of Laboratory Animal Resources (1996), National Research Council, and approved by the National Cancer Institute Animal Care and Use Committee. Experiments were performed within 4 weeks of their arrival at the facility. Their body weight measured before the experiments was in the range 25-28 g. A squamous cell carcinoma was implanted and grown on the right hind leg for a week.

Mice were anesthetized by isoflurane (1.5%) in medical air (700 mL/min). Both tumor and normal legs were placed on special mouse holder and fixed with adhesive tapes on the divider between both legs. The mouse was set in the 25×25 mm (diameter×length) 11 loop parallel coil resonator (Devasahayam, J. Magn. Reson., 142, 168-176 (2000)). The tail vein was cannulated for the injection of nitroxyl contrast agent. Data acquisition was started simultaneously with the injection of the nitroxyl contrast agent (1.8 μmol/g b.w., i.e. 6.0 μL/g b.w. of 300 mM solution). EPRI data acquisition was carried out using home build 300 MHz CW EPR imager (Koscielniak, Rev. Sci. Inst., 71, 4273-7281 (2000)). Twelve projections were obtained every 1.85 min. Other EPR conditions were as follows: microwave frequency=300 MHz, microwave power=2.5 mW, field modulation frequency=13.5 kHz, field modulation amplitude=2.0 Gauss, time constant=0.03 s, sweep width=15 Gauss, scan time=8 s, and the magnitude of the field gradient was 2.5 Gauss/cm. EPR image was reconstructed on 128×128 matrix by filtered back-projection with Shepp-Logan filter. FOV (field of view) was 6×6 cm.

In vivo EPR spectroscopic measurement of the nitroxyl probe were obtained. Mice were anesthetized and placed on special mouse holder and fixed with adhesive tapes. The single loop surface coil (7.3 mm i.d.) was placed on the normal or the tumor leg. EPR signal was measured by CW EPR at 700 MHz. EPR conditions were as follows: microwave frequency=700 MHz, microwave power=10 mW, field modulation frequency=13.5 kHz, field modulation amplitude=0.3 Gauss, time constant=0.03 s, sweep width=60 Gauss, scan time=8 s. The center line of the triplet was repeatedly obtained every 20 s for 20 min.

MRI and pulse sequence measurements were also obtained. MRI measurements were performed at 4.7 T controlled with ParaVision®3.0.1 (Bruker BioSpin MRI GmbH, Rheinstetten, Germany). To perform T₁ and T₂ mapping, spin echo images were obtained using a multi-slice multi-echo (MSME) sequence with 2 different TRs (repetition time: 4000 and 800 ms) and a 16 echo train with 15 ms echo times. The scan time for a T₁ and T₂ mapping image set (N_{EX}=1) by the MSME sequence was 10 min. SPGR (also referred to as gradient echo fast imaging, GEFI) (TR=75 ms, TE=3 ms, FA=45°. N_{EX}=2) was employed to observe T₁ effect. The scan time for an image set (which included 2 slices) by the SPGR sequence was 20 s. Other common image parameters are as follows: image resolution was 256×256, FOV was 3.2×3.2 cm, and slice thickness was 2.0 mm. Number of slice was 2.

In the MRI measurements, mice were anesthetized by isoflurane (1.5%) in medical air (700 mL/min) and secured on a special mouse holder by adhesive skin tape, stomach side down. A breathing sensor (SA Instruments, Inc., NY) was placed on the mouse's back. A non-magnetic temperature probe (FISO, Quebec, Canada) was inserted in the mouse rectum. The tail vein was cannulated for the injection of contrast agent. Then, the mouse was placed in MR resonator, which was previously warmed up by hot water cycling pad. The resonator unit including the mouse was placed in the 4.7 T magnet. The MR measurements were started after the mouse's body temperature came up to 37° C. The mouse body temperature was kept at 37±1° C. during experiment. Prior to the experiments, MSME based T₁ and T₂ mappings were observed. The SPGR based T₁ enhanced image data sets were repeatedly scanned for 20 min. The 1.5 μmol/g b.w. 3CP was injected from tail vein cannulation 2.0 min after starting scan.

The EPRI and MRI data were analyzed using the ImageJ software package (a public domain Java image processing program inspired by NIH Image that can be extended by plug-ins, http://rsb.info.nih.gov/ij/). T₁ and T₂ mappings were calculated using a plug-in (MRI analysis calculator, Karl Schmidt, HypX Laboratory, Brigham and Women's Hospital) available in ImageJ.

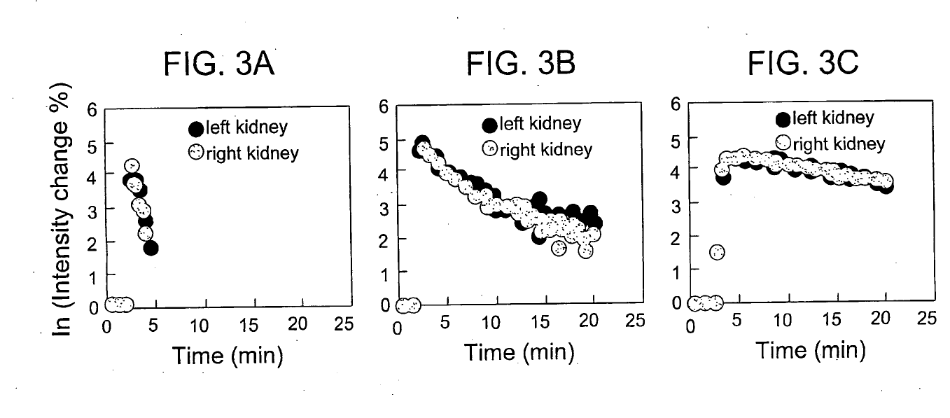

A time course of CW EPR imaging after injection of a nitroxyl contrast agent was determined. Both tumor and normal legs are clearly obtained in each image and the image intensities of both legs are gradually decreased with time. However, any detail of anatomical structure of mouse legs cannot be distinguished in those EPR images. The semi-logarithmic values of the averaged image intensities in the ROIs were plotted with time after injection (FIG. 3). Image intensity once went up and had a peak then began decreasing. The normal leg showed slight delay to reach maximum intensity. A decay rate was obtained from the linear part after peak by the least squares method. Signal decay in the tumor leg was faster than the normal leg. The maximum intensity of the normal leg was smaller than the tumor leg.

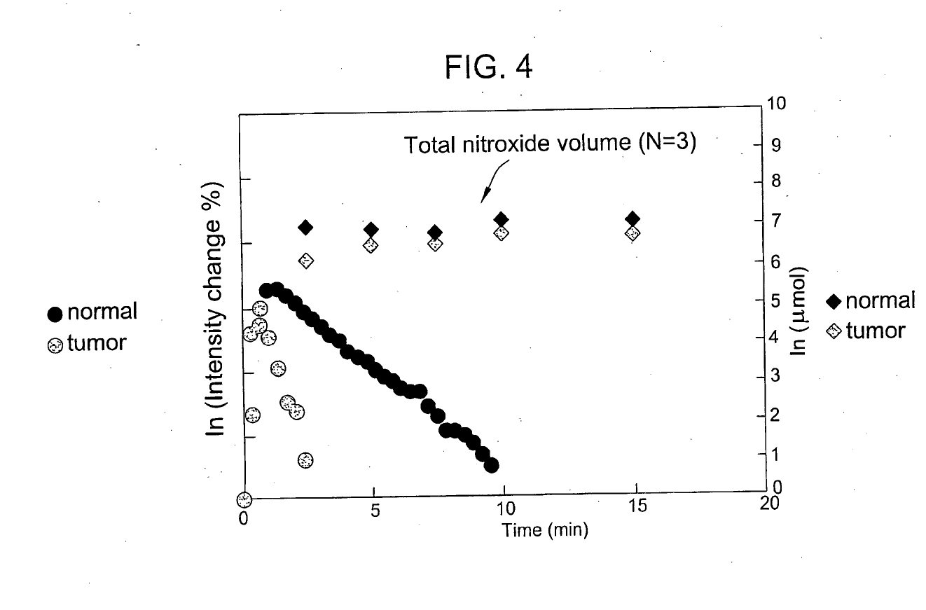

FIG. 4 shows the typical decay profiles obtained by EPR spectroscopic measurement using surface coil resonator worked on 700 MHz. Decay profiles showed similar decay patterns as that obtained by EPRI. Decay rates were obtained from the linear part from 7.5 min to end of measurement (20 min) by minimum square method. Signal decay in the tumor leg was faster than the normal leg. However, difference of the maximum signal intensity between the normal and the tumor legs are smaller than the results from EPRI. Decay constants of both ROIs were obtained by the least squares method. Decay constants of both normal and tumor legs were larger than the results from EPRI.

Two coronal slices (2 mm thickness) including center part of tumor are selected carefully. ROI-1 and ROI-2 were decided based on T₂ mapping. SPGR based T₁-weighted images showed increasing intensity after administration of nitroxyl contrast agent. A time sequence of total 60 SPGR images was obtained during 20 min scan. Therefore, each image (including 2 slices) was obtained every 20 sec. The initial 6 images (obtained before injection) were averaged. Then, every image was divided by the averaged initial image.

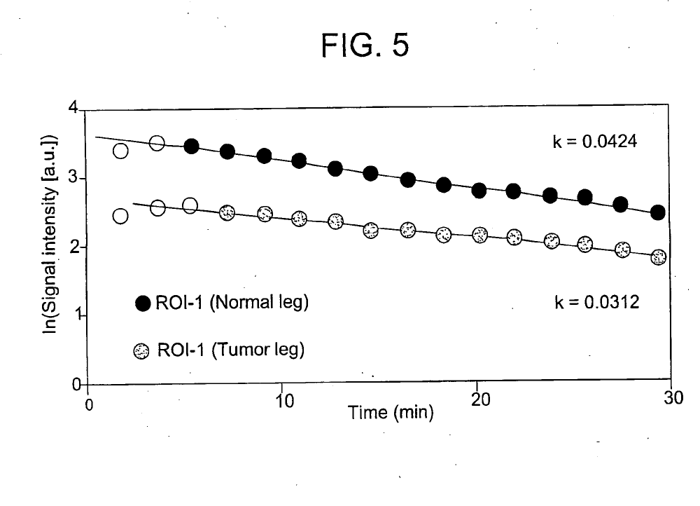

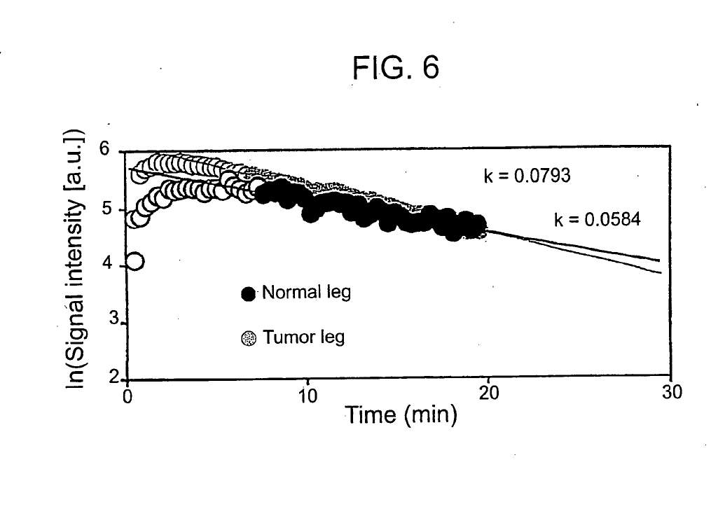

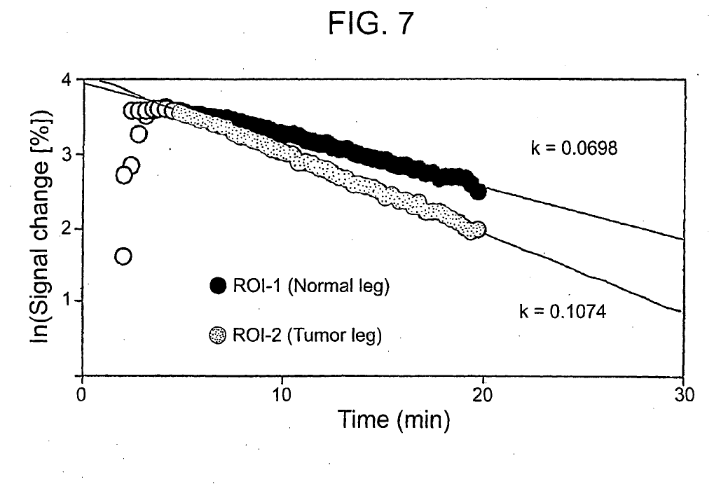

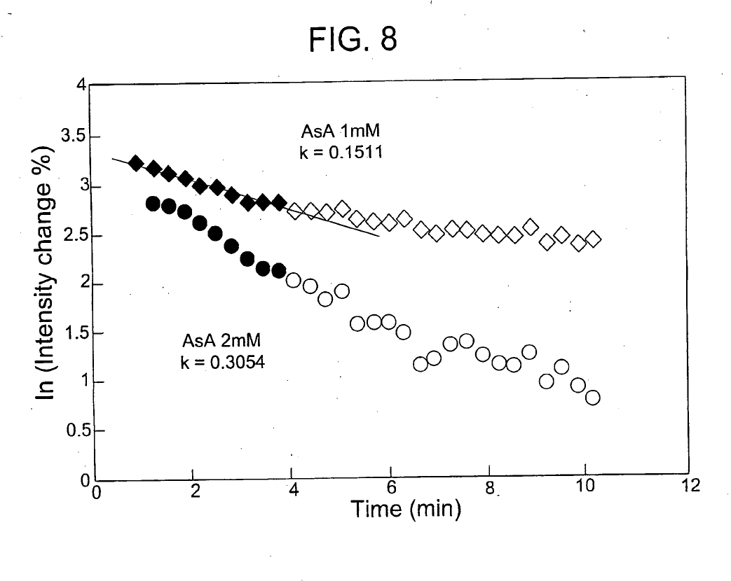

SPGR image intensity immediately went up nearly 60% and gradually decreased. The normal tissue shows slight delay to reach maximum intensity. The image obtained 0.5 min after injection showed signal increase only in tumor tissue. However, the image obtained 1.8 min after injection showed similar signal level in both the tumor and normal tissues. FIG. 5 shows semi-logarithmic plots of the averaged percent difference in the ROIs. Decay constants values of both ROIs were obtained by the least squares method. Decay rate in the tumor leg was higher than the normal leg.

The decay constant of the nitroxyl contrast agent in the tumor and normal tissues of the mouse obtained from in vivo EPR spectroscopic measurement, EPRI, and MRI are summarized in Table 3.

TABLE 3

| | | Normal Leg | | Tumor Leg |
|---|---|---|---|---|
| | Normal | Decay Rate | Tumor Leg | Decay Rate |
| Technique | Leg (n) | (min⁻¹) | (n) | (min⁻¹) |
| EPRI | 4 | 0.0356 ± 0.0122 | 4 | 0.0468 ± 0.0086 |
| EPRS | 10 | 0.0551 ± 0.0059 | 10 | 0.0669 ± 0.0144* |
| MRI | 3 | 0.0766 ± 0.0070 | 3 | 0.1073 ± 0.0057* |

Values indicated as mean ± SD. n indicates number of experiments.
*indicates significances between the normal leg and the tumor leg by p < 0.05.

All methods showed a faster decay in tumor leg. Values of decay constants estimated by MRI were the largest among these methods.

The foregoing demonstrates that MRI techniques using nitroxyl contrast agents provide reliable information regarding redox status of a tumor tissue and normal tissue.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments can become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for determining the redox status of a region of interest in an animal tissue comprising:
a) administering a nitroxyl contrast agent to the region of interest,
b) obtaining a magnetic resonance image of the region of interest, and
c) determining the amount of reduced nitroxyl contrast agent in the region of interest and determining the redox status of the region of interest.

2. The method of claim 1, comprising determining the decay rate of the nitroxyl contrast agent in the region of interest.

3. The method of claim 2, wherein the decay rate is calculated as a change in magnetic resonance image intensity as a function of time.

4. The method of claim 1, wherein the region of interest is selected from the group consisting of normoxic tissue and hypoxic tissue.

5. The method of claim 4, wherein the hypoxic tissue is a tumor tissue.

6. The method of claim 4, wherein the normoxic tissue is adjacent to the hypoxic tissue.

7. The method of claim 1, wherein the animal tissue is a human tissue.

8. The method of claim 1, comprising obtaining at least one magnetic resonance image of the region of interest prior to administering the nitroxyl contrast agent to the region of interest.

9. The method of claim 8, wherein the redox status of the animal tissue is determined by comparing the magnetic resonance images obtained at one or more time points after the administration of the nitroxyl contrast agent.

10. The method of claim 9, wherein the magnetic resonance image is obtained by a spoiled gradient echo (SPGR) magnetic resonance imaging technique.

11. The method of claim 1, wherein the nitroxyl contrast agent is selected from the group consisting of 3-carbamoyl-2,2,5,5-tetramethyl-1-pyrrolidine-N-oxyl, 3-carboxy-2,2,5,5-tetramethyl-1-pyrrolinyloxy, 2,2,6,6-tetramethyl-4-piperidinol-N-oxyl, N-d16-triacetoneamine-N-oxyl, and triacetonamine-N-oxyl, and any combination thereof.

12. The method of claim 1, wherein the magnetic resonance image is obtained of a region of interest in a normoxic tissue and region of interest in a hypoxic tissue and wherein the normoxic tissue is situated adjacent to the hypoxic tissue and wherein the redox status of the normoxic tissue and hypoxic tissue are determined.

13. The method of claim 12, wherein the hypoxic tissue is a tumor tissue.

14. A method for diagnosing a tumor in a region of interest in an animal tissue comprising:
a) administering a nitroxyl contrast agent to an animal tissue whose region of interest is to be monitored,
b) obtaining a magnetic resonance image of the region of interest,
c) obtaining a magnetic resonance image of a tissue adjacent to a region of interest,
d) determining the amount of reduced nitroxyl contrast agent in the tissue adjacent to the region of interest,
e) determining the amount of reduced nitroxyl contrast agent in the region of interest and determining the redox status of the tissue adjacent to the region of interest relative to the redox status of the region of interest, and
f) diagnosing whether there is a tumor present based on the redox status of the region of interest.

15. The method of claim 14, comprising determining the decay rate of the nitroxyl contrast agent in the region of interest.

16. The method of claim 15, wherein the decay rate is calculated as a change in MRI image intensity as a function of time.

17. The method of claim 16, comprising determining the redox status of the region of interest based on the decay rate of the nitroxyl contrast agent in the region of interest.

18. The method of claim 17, comprising determining a time at which there is the greatest difference in the amount of reduced nitroxyl contrast agent between the region of interest and the tissue adjacent to the region of interest.

19. (canceled)

20. A method for determining a cancer treatment protocol comprising:
a) administering a nitroxyl contrast agent to a subject with a tumor,
b) obtaining a magnetic resonance image of the tumor,
c) obtaining a magnetic resonance image of a tissue adjacent to the tumor,
d) determining the amount of nitroxyl contrast agent in the tumor,
e) determining the amount of nitroxyl contrast agent in the tissue adjacent to the tumor, and
f) determining the difference in the amount of nitroxyl contrast agent in the tumor compared with the amount of nitroxyl contrast agent in the tissue adjacent to the tumor to determine a time suitable to administer a dose of radiation.

21. The method of claim 20, comprising determining the decay rate of the nitroxyl contrast agent in the region of interest.

22. The method of claim 21, wherein the decay rate is calculated as a change in MRI image intensity as a function of time.

23. The method of claim 22, comprising determining the redox status of the region of interest based on the decay rate of the nitroxyl contrast agent in the region of interest.

24. The method of claim 23, wherein the time to administer a dose of radiation treatment is determined to be when there is the greatest difference in the amount of reduced nitroxyl contrast agent in the tumor and the tissue adjacent to the tumor.

25. The method of claim 24, wherein the nitroxyl contrast agent is selected from the group consisting of 3-carbamoyl-2,2,5,5-tetramethyl-1-pyrrolidine-N-oxyl, 3-carboxy-2,2,5,5-tetramethyl-1-pyrrolinyloxy, 2,2,6,6-tetramethyl-4-piperidinol-N-oxyl, N-d16-triacetoneamine-N-oxyl, and triacetonamine-N-oxyl, and any combination thereof.
